(19) United States
(12) Patent Application Publication
(10) Pub. No.: US 2007/0055334 A1
(43) Pub. Date: Mar. 8, 2007

(54) Cardiac lead and stylet assembly

(75) Inventors: Paul C. Haldeman, Murrieta, CA (US); Zachary S. Wilson, Wildomar, CA (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/209,422

(22) Filed: Aug. 23, 2005

(51) Int. Cl. A61N 1/05

(52) U.S. Cl. 607/122

(57) ABSTRACT

A cardiac lead and stylet assembly for facilitating placement of cardiac leads for use in combination with cardiac rhythm management devices. The assembly includes a cardiac lead having a lumen therethrough and stylet having a core sized to extend through the lumen. The stylet includes a substantially helical coil sized and adapted to contact and threadingly engage an inner wall of the lead at a reduced diameter portion of the lumen. The stylet coil is also adapted to be threadingly disengaged from the lead to facilitate removal of the stylet without dislodging the lead from a desired implantation site in the patient.

### TECHNICAL FIELD

The present invention relates to the implantation and placement of cardiac leads for use in combination with cardiac rhythm management devices (e.g., pacemakers or defibrillators). More specifically, the invention relates to a cardiac lead/stylet configuration adapted to facilitate lead placement and stylet removal without dislodging the lead from the implantation site.

### BACKGROUND

Implantable medical devices for treating irregular contractions of the heart with electrical stimuli are well known in the art. Some of the most common forms of such implantable devices are defibrillators and pacemakers. Various types of electrical leads for defibrillators and pacemakers have been suggested, many of which are placed transvenously. Such leads are introduced into the patient's vasculature at a venous access site and travel through veins to the sites where the leads' electrodes will be implanted or otherwise contact coronary tissue. Transvenously-placed leads are typically implanted in the endocardium (the tissue lining the inside of the heart) of the right atrium or ventricle, or alternatively, in the branch vessels of the coronary venous system. Such leads include various means to facilitate fixation to the implantation site.

To assist in lead placement, a physician may use devices such as a guide wire and/or guide catheter to navigate a patient's vasculature and to position the lead in its desired location. The lead follows either over the guide wire or within the catheter to the implantation site. Because of the flexible nature of leads, physicians also frequently utilize a stylet inserted into the lead's central lumen to provide shape and steerability to the lead and to assist the physician in fixing the lead's distal end at the patient's implantation site. In addition, physicians may utilize a stabilizing, or “finishing,” wire which, like a stylet, is inserted into the lead's lumen and is used to hold the lead in place during removal of the guide catheter. As used herein, the term “stylet” shall also refer to such stabilizing wires and the like. The stylet is removed after lead placement and fixation is achieved, while the lead remains in place. Because the stylet is designed to engage the lead during lead placement, it can become stuck in the lead's lumen. As a result, removal of the stylet after lead placement can cause the lead to be dislodged or dislocated from the desired implantation site.

Accordingly, there is a need in the art for a stylet or wire adapted to assist the physician in navigating the patient's vasculature and implanting the lead distal end at the desired patient location, yet at the same time be easily removable without dislodging the lead from the implantation site.

### SUMMARY

The present invention, according to one embodiment, is a cardiac lead assembly including an intravenous lead and a stylet. The lead includes a conductor, an electrode, and has an inner wall that defines a lumen extending through at least a portion of the lead. The lumen includes a reduced diameter portion. The stylet has a core that is sized to extend through at least a portion of the lumen, and a substantially helical stylet coil around a portion of the core. Each end of the stylet coil is attached to the stylet core, and the stylet coil has an outer diameter that is greater than the lumen diameter at the reduced diameter portion. The stylet coil is designed to threadingly engage the inner wall when the stylet is rotated.

The present invention, according to another embodiment, is a stylet for use with a cardiac lead having an inner wall that defines a lumen with a reduced diameter portion. The stylet includes a core sized to extend through at least a part of the lumen, and a helical stylet coil around the core. Each end of the stylet coil is attached to the stylet core, and the stylet coil may threadingly engage the inner wall of the lead lumen reduced diameter portion. The stylet also includes a hub attached to one end of the core, and a flexible portion at the other end that is substantially more flexible than the other portion of the stylet.

The present invention, in yet another embodiment, is a method of placing a cardiac lead at an implantation site in a patient. The method includes advancing a guide catheter through a patient's vasculature until its distal end is at a predetermined location, providing a cardiac lead having an inner wall that defines a lumen extending through at least a portion of the lead, inserting and advancing a stylet into the lumen, threadingly engaging the lead with the stylet, and advancing the cardiac lead with stylet through the guide catheter to the implantation site.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic view of a lead/stylet assembly deployed in a heart and parts of the vascular system, according to one embodiment of the present invention.

FIG. 2 is a perspective view of a lead for use in delivering a therapy, according to one embodiment of the present invention.

FIG. 3 is a partial cross-sectional view showing a distal portion of the lead depicted in FIG. 2.

FIG. 4 is a schematic view of a stylet according to one embodiment of the present invention.

FIGS. 5A-5B depict partial cross-sectional views of a lead/stylet assembly showing the stylet of FIG. 4 inserted into the lead of FIG. 2, according to one embodiment of the present invention.

FIGS. 6A-6C depict alternative lead designs according to various embodiments of the present invention.

FIGS. 7A-7B depict alternative stylet designs according to various embodiments of the present invention.

FIG. 8 is a flowchart illustrating the use of the lead and stylet assembly according to one embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

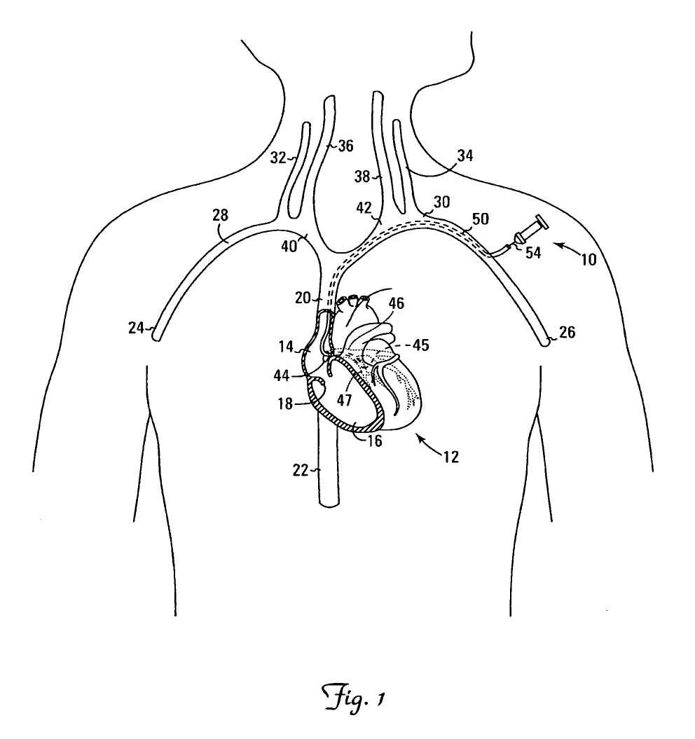

FIG. 1 shows a cardiac lead and stylet assembly 10 deployed in a human heart 12 according to an embodiment of the present invention. As shown in FIG. 1, the heart 12 includes a right atrium 14 and a right ventricle 16 separated by a tricuspid valve 18. During normal operation of the heart 12, deoxygenated blood is fed into the right atrium 14 through the superior vena cava 20 and the inferior vena cava 22. The major veins supplying blood to the superior vena cava 20 include the right and left axillary veins 24 and 26, which flow into the right and left subclavian veins 28 and 30. The right and left external jugular 32 and 34, along with the right and left internal jugular 36 and 38, join the right and left subclavian veins 28 and 30 to form the right and left brachiocephalic veins 40 and 42. The right and left brachiocephalic veins 40 and 42 combine to flow into the superior vena cava 20.

The lead/stylet assembly 10, shown in FIG. 1, enters the vascular system through a wall of the left subclavian vein 30, extends through the left brachiocephalic vein 42 and the superior vena cava 20, and enters the right atrium 14. As further shown, in one embodiment, the lead/stylet assembly 10 then enters the coronary sinus ostium 44 so that a distal portion 45 of the assembly 10 extends within the coronary sinus 46 and into a lateral branch 47 off of the coronary sinus 46. Alternatively, the assembly 10 may be positioned so that the distal portion 45 is located in the right atrium 14 or right ventricle 16. In other embodiments of the present invention, the lead/stylet assembly 10 may enter the vascular system through the right subclavian vein, the left axillary vein 26, the left external jugular 34, the left internal jugular 38, or the left brachiocephalic vein 42. As shown in FIG. 1, the lead/stylet assembly 10 includes a lead 50 and a stylet 54 inserted therein.

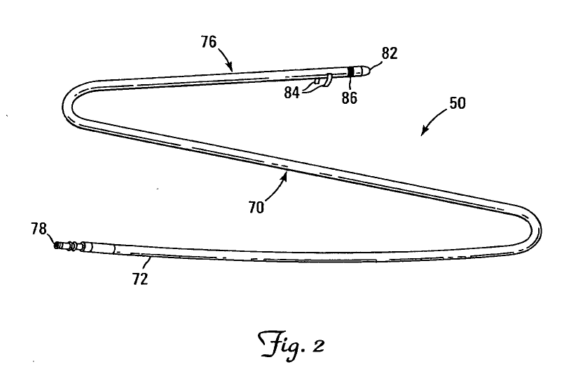

FIG. 2 is a perspective view of a lead 50 for use in delivering a therapy, according to one embodiment of the present invention. As shown in FIG. 2, the lead 50 includes, in one embodiment, an elongated main body 70 having a proximal portion 72 and a distal portion 76. The lead proximal portion 72 has a proximal end 78, and the lead distal portion 76 terminates at a lead distal tip 82. The lead 50 may also include means, such as tines 84, for facilitating fixation of the distal portion 76 at or near desired tissue in a patient, as well as one or more electrodes 86 for delivering a therapy to the patient. The lead body 70 may be constructed of a flexible, electrically insulative material.

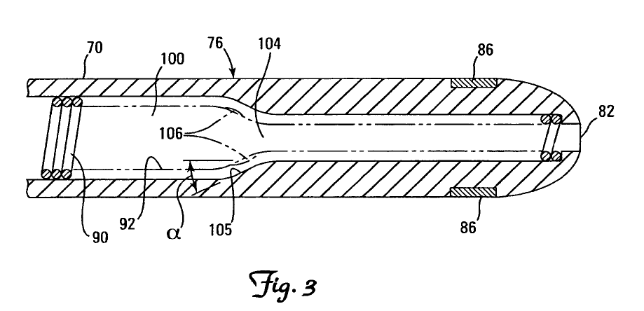

FIG. 3 is a partial cross-sectional view of the lead 50 depicted in FIG. 2 showing the lead distal portion 76. As shown in FIG. 3, the lead 50 includes, in one embodiment, an insulated conductor 90 encapsulated by the lead body 70, and a lumen 100 defined by an inner wall 92. The lumen 100 extends from the lead proximal end 78 to the distal tip 82 (see FIG. 2), and includes a reduced diameter portion 104 located within or near the distal portion 76. In the embodiment shown in FIG. 3, the conductor 90 is shown as a tightly spaced coil. Alternatively, the conductor 90 may be in the form of a conductive wire, thin ribbon, or a plurality of conductive wires formed as a cable. In FIG. 3, the wall 92 of the lumen 100 is formed by the inner surface of the conductor coil 90, although in other embodiments, a separate covering may form the wall 92. Note that in FIG. 3 (as well as other figures depicting a cross-sectional view of the lead 50), the dashed line representing the inner wall 92 is intended to represent any and all such embodiments and configurations of the inner wall 92. Additionally, in the embodiment shown in FIG. 3, the lumen reduced diameter portion 104 extends through the distal tip 82, although this is not a requirement of the present invention, as will be shown in more detail below. In one embodiment, the reduced diameter portion 104 includes a taper 105 having a taper angle α. The reduced diameter portion 104 may optionally include one or more substantially helical female threads 106.

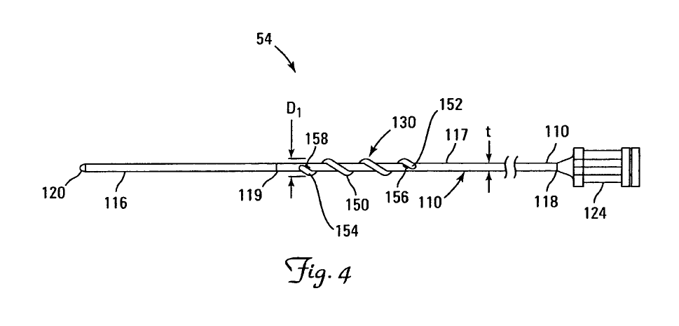

FIG. 4 is a schematic view of a stylet 54 according to one embodiment of the present invention. As can be seen in FIG. 4, the stylet 54 includes, in one embodiment, a proximal portion 110 and a distal portion 116. In one embodiment, the stylet distal portion 116 may be substantially more flexible than the stylet proximal portion 110, although this is not a requirement of the present invention. The stylet proximal portion 110 includes a stylet core 117 having a proximal end 118 and a distal end 119, and a stylet coil 130 disposed about a portion of the stylet core 117. The stylet distal portion 116 is attached to the stylet core 117 at the core distal end 119 and terminates in a distal tip 120. The stylet distal portion 116 and core 117 are generally sized and shaped to extend within the lumen 100 of the lead 50 and to facilitate navigation of the patient's vasculature to the desired treatment location. The stylet 54 may include a hub 124 located at and coupled to the core proximal end 118 for facilitating manipulation of the stylet 54 by the physician.

As shown in FIG. 4, the stylet coil 130 is generally helical and is formed from a wire 150 and has a first end 152 and a second end 154. The stylet coil 130 may be formed using any method known in the art.

In one embodiment, the stylet coil first end 152 is attached to the stylet core 117 by a first joint 156, and the stylet coil second end is attached to the stylet core 117 by a second joint 158. In another embodiment, the stylet coil 130 may also be attached to the stylet core 117 intermittently and/or along substantially the entire length of the stylet coil 130. Any suitable joining method known in the art (e.g., soldering, welding or brazing) may be used to attach the stylet coil 130 to the stylet core 117. In another embodiment, the stylet coil 130 is formed integral to the stylet core 117 (e.g., by machining the stylet coil 130 into the stylet core 117 and then machining the stylet core 117 to the desired final dimensions). In yet another embodiment, the stylet coil 130 may be formed or attached to a sleeve (not shown) which is subsequently slid over and attached to the stylet core 117.

The diameters of the stylet core 117 and the wire 150 used to form the stylet coil 130 generally determine an outer diameter D1 of the stylet coil 130. In one embodiment, the stylet core 117 may have a constant thickness t ranging from about 0.004″ to about 0.015″. In another embodiment, the stylet core 117 may be tapered distally, with a largest thickness t at the core proximal end 118 of from about 0.006″ to about 0.015″, and a thickness t at the core distal end 119 of from about 0.004″ to about 0.012″. In one embodiment, the coil wire 150 may consist of a round, constant diameter wire ranging from about 0.004″ to about 0.010″ in diameter. In one exemplary embodiment, the coil wire 150 is about 0.006″ in diameter and the stylet core 117 has a thickness t of about 0.010.″ In another embodiment, the coil wire 150 is about 0.008″ in diameter.

In one embodiment, the stylet core 117 and the coil wire 150 are stainless steel, although other materials may also be suitable, as will be apparent to those skilled in the art. The stylet core 117 may optionally be coated with a polymer such as PTFE along all or part of its length.

In one embodiment, the stylet distal portion 116 may be substantially more flexible than the stylet proximal portion 110, which facilitates navigation of the lead 50 with stylet 54 through the patient's vasculature. In one embodiment of the stylet 54, the flexible stylet distal portion 116 may be formed from a thin wire (e.g., from about 0.0022″ diameter to about 0.0028″ diameter) wound in a tight spiral. Additionally, the flexible stylet distal portion 116 may be pre-shaped (e.g., in a J-shape) to further aid in vessel navigation and subselection. The stylet distal portion 116 may be made of the same materials as the stylet core 117, or of different materials as are known in the art. The stylet distal portion 116 may be attached to the stylet core 117 using any methods known in the art (e.g., soldering, welding or brazing).

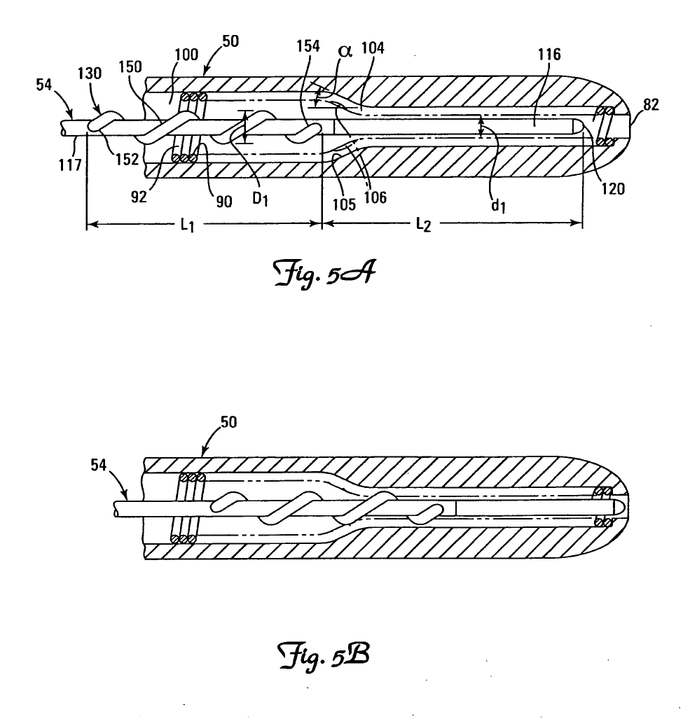

FIG. 5A is a partial cross-sectional view of the lead/stylet assembly 10 showing the lead 50 with the stylet 54 inserted into the lumen 100, according to one embodiment of the present invention. As can be seen in FIG. 5A, the stylet coil outside diameter D1 is greater than a lumen diameter d1 at the reduced diameter portion 104. Thus, the stylet coil 130 is sized and shaped to contact and engage the inner wall 92 at the reduced diameter portion 104, thereby inhibiting further advancement of the stylet 54 through the lumen 100.

Because of its generally helical shape, the stylet coil 130 may threadingly engage the inner wall 92 by threading itself into the reduced diameter portion 104. In one embodiment, the stylet coil 130 threads itself into the reduced diameter portion 104 by deforming the inner wall 92 in a helical pattern as it advances forward. This threaded engagement is further promoted if clockwise torque along with a slight distal force are applied to the stylet 54 by the physician. In addition, if the stylet 54 becomes stuck in the lead 50, applying slight counter-clockwise torque (i.e., about 1 rotation) will “unscrew” the stylet coil 130 from the wall 92, permitting the stylet 54 to be easily removed from the lead 50 without excessive force, which could otherwise dislodge the lead 50 from the desired implantation site. FIG. 5B depicts the style 54 threadingly engaged with the lead 50.

The outer diameter D1 of the stylet coil 130 may range from about 0.012″ to about 0.035″. The lumen reduced diameter portion 104 may have a diameter d1 of from about 0.008″ to about 0.032″. In general, the ratio of diameters D1 and d1 of the stylet coil 130 and reduced diameter portion 104 are selected so as to provide an interference fit between the stylet coil 130 and the lumen wall 92 at the reduced diameter portion 104. Thus, in one exemplary embodiment, the stylet coil diameter D1 is about 0.022″ and the diameter d1 of the lumen reduced diameter portion is about 0.018″. In another exemplary embodiment, the stylet coil diameter D1 is about 0.024″ and the diameter d1 of the lumen reduced diameter portion is about 0.021″.

The stylet coil 130 should have a sufficient number of “turns” (i.e., complete revolutions) of the wire 150 about the stylet core 117 to ensure positive fixation between the stylet 54 and the lead 50. In one exemplary embodiment, the stylet coil 130 has two and one-half turns where the stylet coil outer diameter D1 is 0.022 inches and the lumen diameter d1 at the reduced diameter portion is 0.018 inches. In other embodiments, the stylet coil 130 may have more or fewer turns. In one embodiment, the stylet coil 130 has three turns over an axial length L1 of between about 0.15 inches and about 0.40 inches.

In yet another embodiment, the stylet coil 130 is not formed from a continuous length of wire, but is instead formed from wire segments arranged in a generally helical pattern along the stylet core 117.

As shown in FIG. 5A, in one embodiment, the second end 154 of the stylet coil 130 is located at a distance L2 from the stylet distal tip 120, such that the stylet distal tip 120 is generally adjacent to, but does not extend beyond, the lead distal tip 82 when the stylet 54 is fully inserted into the lumen 100. Thus, depending on the dimensions of the corresponding lead 50, the distance L2 may, in various embodiments, range from about 0.50 inches to about 1.20 inches. In one exemplary embodiment, the distance L2 is about 0.70 inches. In another exemplary embodiment, the distance L2 is about 1.0 inches. It should be noted that the stylet coil 130 may be located closer to the core proximal end 118 (see FIG. 4) if the design of the corresponding lead 50 so dictates.

In the embodiment of the lead 50 depicted in FIGS. 3 and 5A, the lumen 100 includes a taper 105. In general, the more gradual the taper 105, the further the stylet coil 130 will engage the lumen inner wall 92, with a resulting increase in fixation between the stylet 54 and the lead 50. In one embodiment, the taper 105 may have a taper angle α of from about 10 degrees to about 60 degrees. In one embodiment, the female thread(s) 106 may be sized and shaped to mate with the stylet coil 130, thereby providing additional fixation of the stylet 54 to the lead 50.

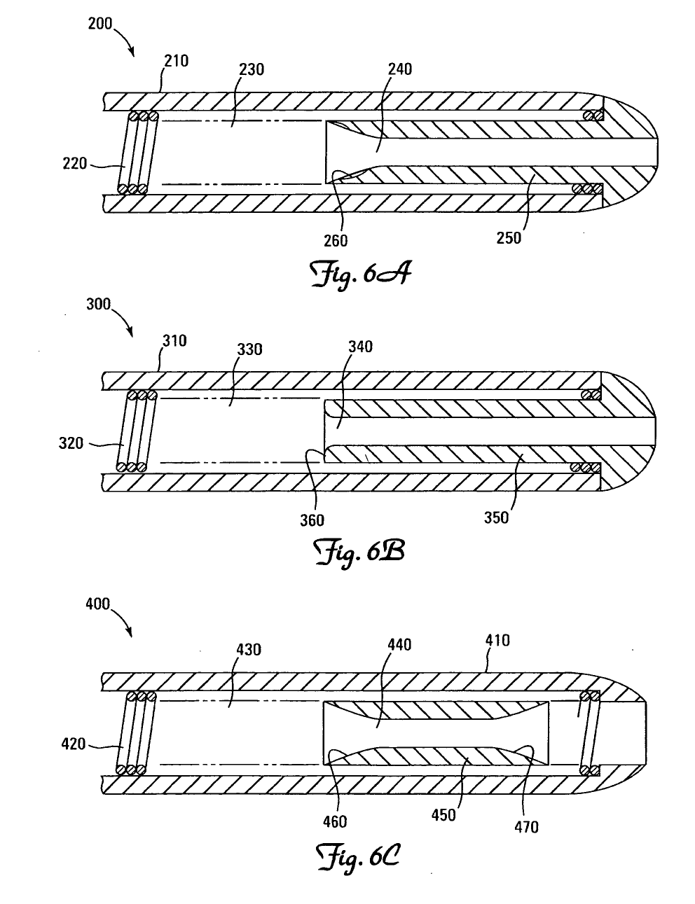

FIG. 6A-6C depict partial cross-sectional views of alternative leads 200, 300, and 400 according to other embodiments of the present invention. In FIG. 6A, the lead 200 includes a body 210, a conductor 220, and a lumen 230 having a reduced diameter portion 240, which, for example, may be formed by an insert 250. The insert 250 may be made from the same material as the body 210 (i.e., silicone or urethane). In one embodiment, the insert 250 includes threads 260 formed to generally match and mate with the stylet coil 130.

Where an insert 250 is used, the coil wire 150 may be a flat wire, which may provide additional fixation of the stylet 54 to the lead 50 due to the relatively sharp edges of the wire 150. In such an embodiment, the insert 250 protects the conductor 90 from possible damage by the stylet coil 130.

In FIG. 6B, the lead 300 includes a body 310, a wire conductor 320, and a lumen 330 having a reduced diameter portion 340 formed by an insert 350. The lumen 330 abruptly changes diameter at step 360.

In FIG. 6C, the lead 400 includes a reduced diameter portion 440 formed from an insert 450 and a distal tip 480. In this embodiment, the reduced diameter portion 440 does not extend to the distal tip 480.

Although the embodiments shown in FIGS. 6A-C use inserts 250, 350 and 450, in other embodiments, the reduced diameter portions 240, 340 and 440 may be formed by the wire conductors 220, 320 and 420, as in the lead depicted in FIG. 2.

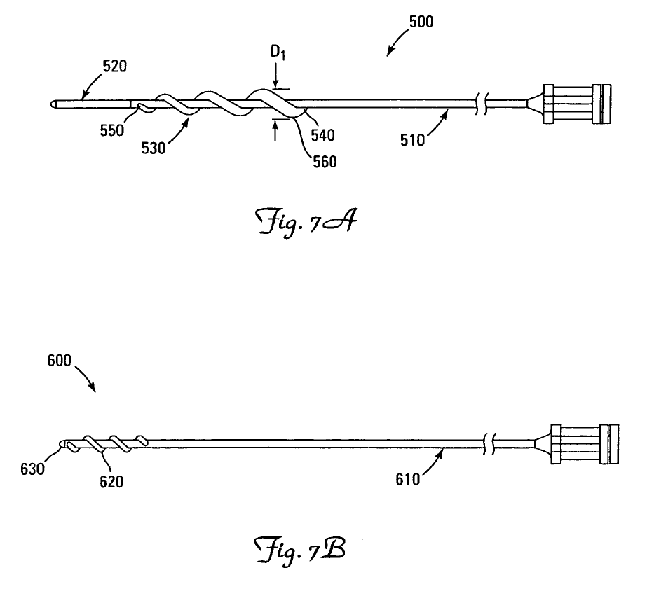

FIGS. 7A-7B depict alternative stylets 500 and 600 according to other embodiments of the present invention. In FIG. 7A, the stylet 500 has a proximal portion 510 and a distal portion 520. The proximal portion 510 includes a stylet coil 530 having a first end 540 and a second end 550. The stylet coil 530 is formed from a coil wire 560 which is tapered, with a larger diameter end constituting the first end 540 of the stylet coil 530. The resulting stylet coil 530 is also tapered, so that the coil diameter D1 near the first end 540 is larger than at the second end 550. This tapered coil may promote positive fixation of the stylet 500 to the lead 50.

In FIG. 7B, the stylet 600 has a stylet core 610 and a stylet coil 620 disposed about the stylet core 610. In this embodiment, the stylet coil 620 forms a distal portion of the stylet, and only a distal tip 630 extends beyond the stylet coil 620. Such a design may, in one embodiment, be used as a stabilizing wire for facilitating removal of a guide catheter after placement of the lead 50.

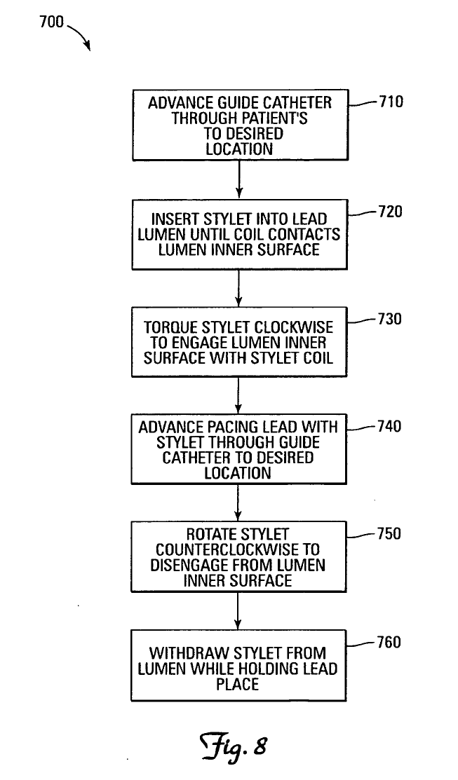

FIG. 8 is a flowchart illustrating the use of the lead 50 and stylet 54 according to one embodiment of the present invention. As shown in FIG. 8, a physician first advances a guide catheter to a desired location in the patient's vasculature (e.g., a coronary vein or the right atrium) (block 710). In one embodiment, the physician may then preload the lead 50 by inserting the stylet 54 into the lumen 100 until the stylet coil 130 contacts the lumen inner wall 92 at the reduced diameter portion 104 (block 720). If additional fixation of the stylet 54 to the lead 50 is desired, the physician may rotate the stylet 54 clockwise (by turning the hub 124) so that the stylet coil 130 threadingly engages the inner wall 92 (block 730). The physician then advances the lead 50 with stylet 54 through the guide catheter until the distal tip 82 is adjacent to the patient's tissue selected for treatment (block 740). The stylet 54 provides the necessary stiffness to the lead 50 to facilitate navigation to the desired location. Additionally, the threaded engagement between the stylet coil 130 and the inner wall 92 permits the physician to apply a proximal force to the lead 50 by pulling on the stylet hub 124. This advantageously allows the physician to retract the lead 50 without applying force directly to the lead proximal end 78, which could otherwise damage the lead 50. Thus if, for example, an initial implantation site is found to be less than optimal, the physician can readily retract the lead/stylet assembly 10 from the initial location and advance it to a new location.

The physician may then disengage the stylet coil 130 from the inner wall 92, by applying reverse torque to the stylet 54 by rotating the hub 124 counterclockwise, thereby unthreading the stylet coil 130 from the inner wall 92 (block 750). The physician may then remove the stylet 54 from the lumen 100 by pulling on the hub 124 and stylet proximal portion 110 while holding the lead 50 in place (block 760). Because the stylet coil 130 has already been disengaged from the inner wall 92, removal of the stylet 54 can be accomplished without dislocating the lead 50 from the desired treatment location.

In addition, where the stylet 54 is used as a stabilizing wire for guide catheter removal and is fully inserted into the lumen 100 of the lead 50, the physician may remove the guide catheter by pulling on the guide catheter while holding the stylet 54 in place. The stylet 54 may then be removed as described above.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A cardiac lead assembly, comprising:
an intravenous lead having a conductor, an electrode, and an inner wall defining a lumen extending through at least a portion of the lead, wherein the lumen includes a reduced diameter portion; and
a stylet having a core sized to extend through at least a portion of the lumen, and a substantially helical stylet coil disposed about a portion of the core, wherein the stylet coil has a first end attached to the core and a second end attached to the core, such that the stylet coil defines a coil outer diameter greater than a lumen diameter at the reduced diameter portion;
wherein the stylet coil is adapted to threadingly engage the inner wall upon rotation of the stylet.

2. The assembly of claim 1 wherein the stylet further comprises a flexible portion attached to the core, wherein the flexible portion is substantially more flexible than the core.

3. The assembly of claim 2 wherein the stylet coil is disposed about the core at a location near the flexible portion.

4. The assembly of claim 3 wherein the stylet flexible portion includes a distal tip, and wherein the stylet coil second end is located between about 0.5 inches and about 1.2 inches from the distal tip.

5. The assembly of claim 4 wherein the stylet coil second end is located about 1.0 inches from the distal tip.

6. The assembly of claim 1 wherein the reduced diameter portion includes a taper.

7. The assembly of claim 6 wherein the taper is formed by the conductor of the cardiac lead.

8. The assembly of claim 6 wherein the taper is formed by an insert.

9. The assembly of claim 1 wherein the reduced diameter portion includes a substantially helical female thread adapted to mate with the stylet coil.

10. The assembly of claim 1 wherein the stylet coil outer diameter is from about 0.012 inches to about 0.035 inches.

11. The assembly of claim 10 wherein the lumen diameter at the reduced diameter portion is from about 0.008 inches to about 0.032 inches.

12. The assembly of claim 11 wherein the stylet coil outer diameter is about 0.022 inches and the lumen diameter at the reduced diameter portion is about 0.018 inches.

13. The assembly of claim 11 wherein the stylet coil outer diameter is about 0.030 inches and the lumen diameter at the reduced diameter portion is about 0.026 inches.

14. The assembly of claim 10 wherein the stylet coil comprises a coil wire having a first wire diameter of from about 0.004 inches to about 0.010 inches.

15. The assembly of claim 14 wherein the first wire diameter is about 0.006 inches.

16. The assembly of claim 14 wherein the core has a thickness of from about 0.004 inches to about 0.015 inches.

17. The assembly of claim 16 wherein the core thickness is about 0.010 inches and the first wire diameter is about 0.006 inches.

18. A stylet for use with a cardiac lead having an inner wall defining a lumen with a reduced diameter portion, the stylet comprising:
a proximal portion including
a core sized and shaped to extend through at least a part of the lumen,
a substantially helical stylet coil disposed about a portion of the core, wherein the stylet coil has a first end attached to the core and a second end attached to the core, and is adapted to threadingly engage the inner wall upon rotation of the stylet, and
a hub attached to a proximal end of the core; and
a distal portion attached to the proximal portion, wherein the distal portion is substantially more flexible than the proximal portion.

19. The stylet of claim 18 wherein the stylet coil is disposed about the core at a location near the distal portion.

20. The stylet of claim 19 wherein the distal portion comprises a distal tip, and wherein the stylet coil second end is located between about 0.50 inches and about 1.20 inches from the distal tip.

21. The stylet of claim 20 wherein the stylet coil second end is located about 1.0 inches from the distal tip.

22. The stylet of claim 18 wherein the stylet coil outer diameter is from about 0.012 inches to about 0.035 inches.

23. The stylet of claim 22 wherein the stylet coil outer diameter is about 0.022 inches.

24. A method of placing a cardiac lead at an implantation site in a patient, the method comprising:
advancing a guide catheter through a patient's vasculature until a distal end thereof is at a predetermined location;
providing a cardiac lead having an inner wall defining a lumen extending through at least a portion of the lead;
inserting and advancing a stylet into the lumen;
threadingly engaging the lead with the stylet; and
advancing the cardiac lead with stylet through the guide catheter to the implantation site.

25. The method of claim 24 further comprising threadingly disengaging the stylet from the lead.

26. The method of claim 24 further comprising removing the guide catheter from the patient's vasculature while using the stylet to hold the cardiac lead at the implantation site.

27. The method of claim 26 further comprising threadingly disengaging the stylet from the lead.
